(19) United States
(12) Patent Application Publication
(10) Pub. No.: US 2024/0111953 A1
(43) Pub. Date: Apr. 4, 2024

(54) EVIDENCE RESULT NETWORK

(71) Applicant: Scinapsis Analytics Inc., dba BenchSci, Toronto (CA)

(72) Inventors: Tom LEUNG, North York (CA); Elvis WIANDA, Oakville (CA); Anshuman SAHOO, Toronto (CA)

(73) Assignee: Scinapsis Analytics Inc., dba BenchSci, Toronto (CA)

(21) Appl. No.: 17/958,142

(22) Filed: Sep. 30, 2022

(51) Int. Cl. G06F 40/284; G06V 30/14; G16H 70/00

(52) U.S. Cl. CPC G06F 40/284; G06V 30/1444; G16H 70/00

(57) ABSTRACT

A method implements an evidence result network. The method includes receiving a file; tokenizing a sentence from the file to generate a set of tokens; processing the set of tokens to generate a tree for the sentence. The method further includes processing the tree to generate a graph for the sentence; processing the graph to identify correspondences between nodes of the graphs and types of entities of an ontology library; and presenting the graph.

### BACKGROUND

Biomedical information includes literature and writings that describe evidence from experiments and research of biomedical science that provides the basis for modern medical treatments. Biomedical information is published in publications in physical or electronic form and may be distributed in electronic form using files. Databases of files of biomedical information provide access to the electronic forms of the publications. A challenge is for computing systems to identify the evidence in files containing biomedical information.

### SUMMARY

In general, in one or more aspects, the disclosure relates to a method implementing an evidence result network. The method includes receiving a file; tokenizing a sentence from the file to generate a set of tokens; processing the set of tokens to generate a tree for the sentence. The method further includes processing the tree to generate a graph for the sentence; processing the graph to identify correspondences between nodes of the graphs and types of entities of an ontology library; and presenting the graph.

In general, in one or more aspects, the disclosure relates to a system implementing an evidence result network. The system includes a graph controller configured to generate a graph and an application executing on one or more servers. The application is configured for receiving a file, tokenizing a sentence from the file to generate a set of tokens, and processing the set of tokens to generate a tree for the sentence. The application is further configured for processing the tree to generate the graph for the sentence, processing the graph to identify correspondences between nodes of the graphs and types of entities of an ontology library, and presenting the graph.

In general, in one or more aspects, the disclosure relates to a method of an evidence result network. The method includes transmitting a request and displaying a graph received in a response to the request. The graph is generated by tokenizing a sentence from a file to generate a set of tokens; processing the set of tokens to generate a tree for the sentence; processing the tree to generate a graph for the sentence; and processing the graph to identify correspondences between nodes of the graphs and types of entities of an ontology library.

Other aspects of the invention will be apparent from the following description and the appended claims.

### BRIEF DESCRIPTION OF DRAWINGS

FIG. 1A, FIG. 1B, and FIG. 1C show diagrams of systems in accordance with disclosed embodiments.

FIG. 2A and FIG. 2B show flowcharts in accordance with disclosed embodiments.

FIG. 3A, FIG. 3B, FIG. 4, FIG. 5, and FIG. 6 show examples in accordance with disclosed embodiments.

FIG. 7A and FIG. 7B show computing systems in accordance with disclosed embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure implement evidence result networks. An evidence result network may be implemented as a graph generated from a file or other scientific evidence by a computing system. The system receives a file (i.e., including but not limited to publications of biomedical information, patents, documents of experimental results originating from external and internal customer sources, etc.) that stores the evidence using text, images, data, etc. The evidence (i.e., the data of a file describing scientific results) is processed to identify the sentences and images in the file. The file may be referred to as a source of evidence of a scientific result. One or more machine learning models may be used to process the sentences and images to generate graphs (evidence result networks) that represent the evidence demonstrated from experiments described by the sentences and images from the file. An ontology library (saved as a collection of data records) is used to identify terms and phrases from the text and images of the file that relate to entities with biomedical meaning. For example, the ontology library may store the names of proteins, diseases, experimentation techniques, etc. The entities from the ontology library may be recognized during the processing of the file to preserve the meaning of terms and phrases from the text and images in the graphs generated by the system.

The machine learning models used by the system may be trained to understand evidence both written and visual. For example, a machine learning model may be trained to recognize and tag entities in biomedical information, defined by the data records of the ontology library, that appear in a sentence. Additional machine learning models (semantic tree generators, image recognizers, etc.) may be trained with biomedical data (text and images) to be customized for biomedical data.

After a file is processed to generate a set of result graphs for the evidence described by the data of the file, the graphs and images from the file may be displayed to a user. For example, a user interested in the relationship between two entities (e.g., a protein and a disease) may locate a file corresponding to a biomedical publication that includes the two entities in a graph generated from a sentence or image from the file. Graphs and images that describe the relationships between the entities may then be displayed to the user.

The figures show diagrams of embodiments that are in accordance with the disclosure. The embodiments of the figures may be combined and may include or be included within the features and embodiments described in the other figures of the application. The features and elements of the figures are, individually and as a combination, improvements to the technology of biomedical information processing and machine learning models. The various elements, systems, components, and steps shown in the figures may be omitted, repeated, combined, and/or altered as shown from the figures. Accordingly, the scope of the present disclosure should not be considered limited to the specific arrangements shown in the figures.

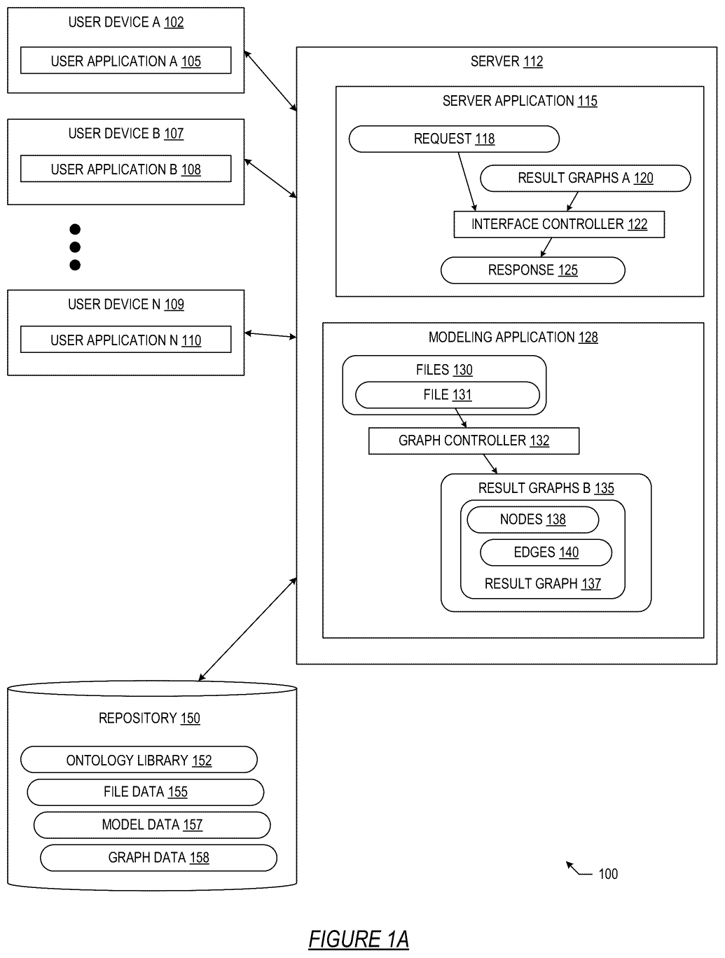

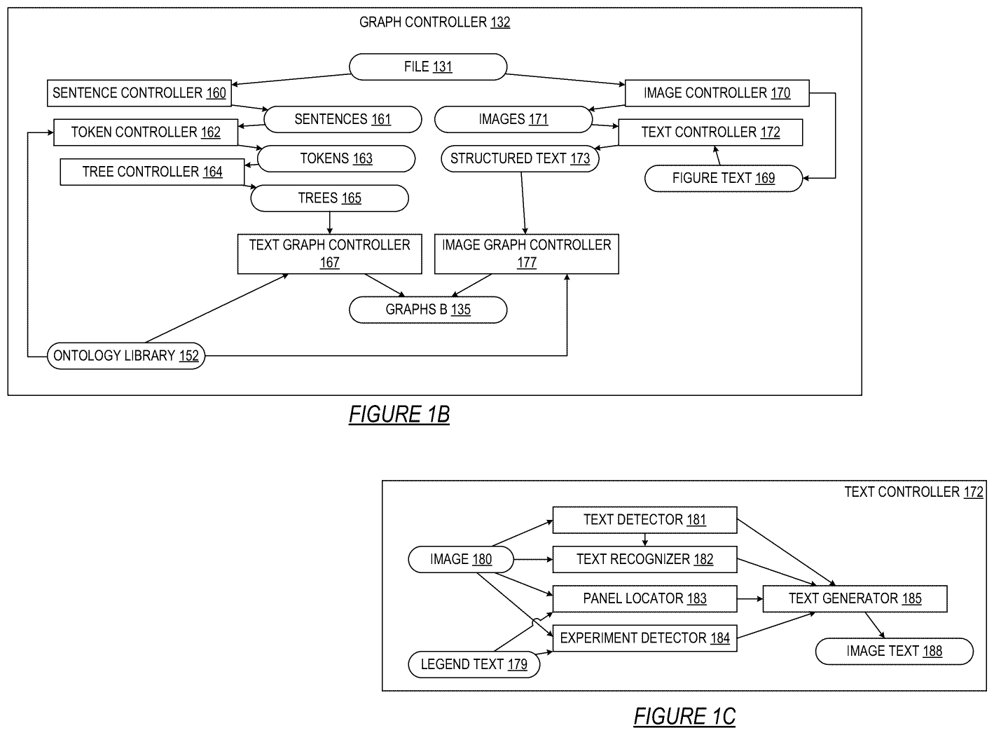

Turning to FIG. 1A, the system (100) implements evidence result networks by converting files (including biomedical publications) to graphs. The system (100) receives requests (e.g., the request (118)) and generates responses (e.g., the response (125)) using the result graphs A (120). The system (100) generates the result graphs A (120) from files (e.g., the file (130)) stored in the file data (155) using multiple machine learning and natural language processing models. The system (100) uses the result graphs A (120) to identify entities specified in the request (118) and generate the response (125). The system (100) may display the result graphs A (120) and the images from the files of the file data (155) to users operating the user devices A (102) and B (107) through N (109). The system (100) includes the user devices A (102) and B (107) through N (109), the server (112), and the repository (150).

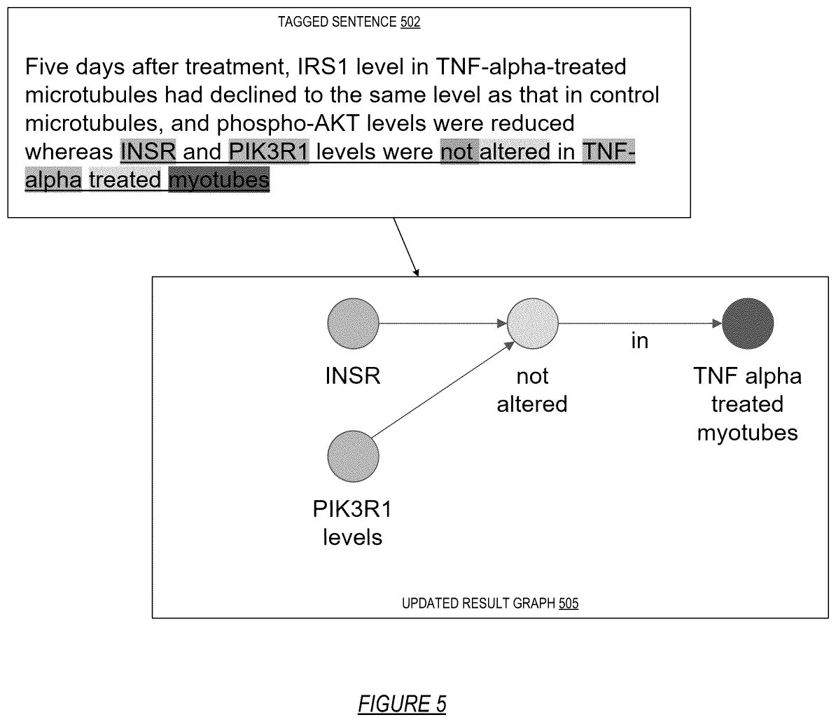

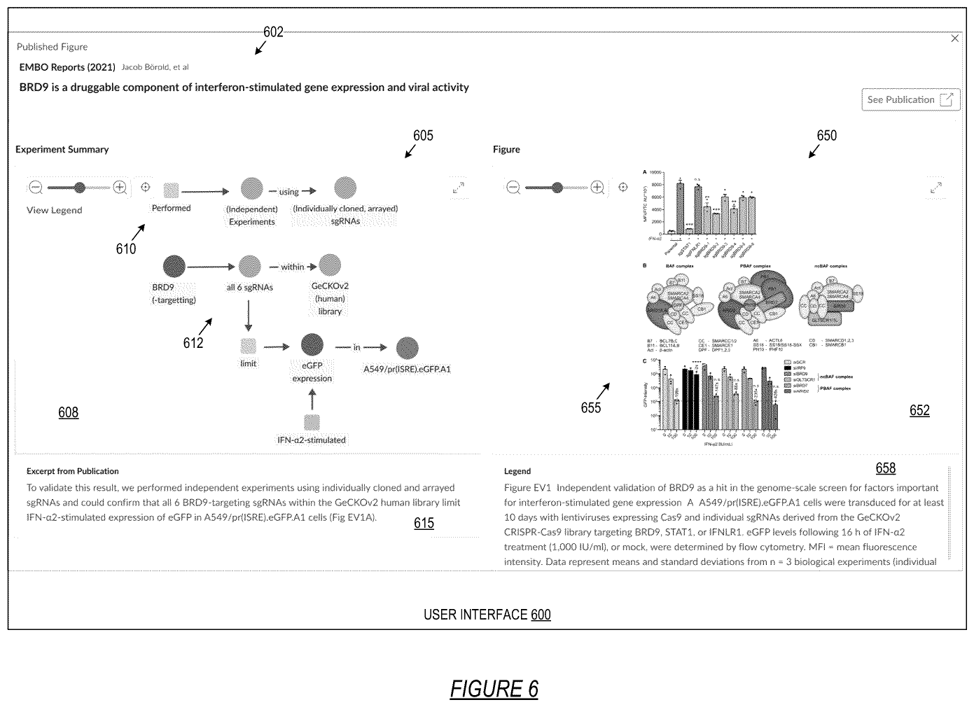

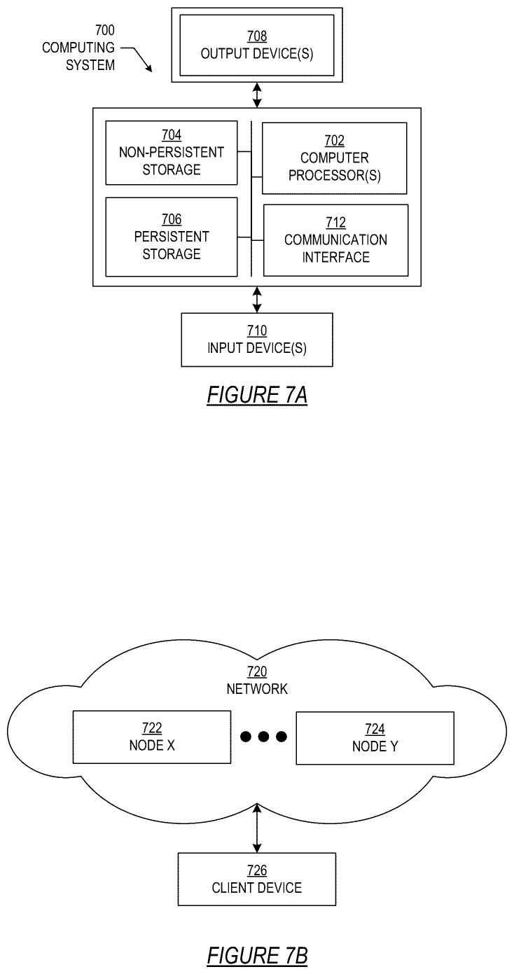

The server (112) is a computing system (further described in FIG. 7A). The server (112) may include multiple physical and virtual computing systems that form part of a cloud computing environment. In one embodiment, execution of the programs and applications of the server (112) is distributed to multiple physical and virtual computing systems in the cloud computing environment. The server (112) includes the server application (115) and the modeling application (128).

The server application (115) is a collection of programs that may execute on multiple servers of a cloud environment, including the server (112). The server application (115) receives the request (118) and generates the response (125) based on the result graphs A (120) using the interface controller (122). The server application (115) may host websites accessed by users of the user devices A (102) and B (107) through N (109) to view information from the result graphs A (120) and the file data (155). The websites hosted by the server application (115) may serve structured documents (hypertext markup language (HTML) pages, extensible markup language (XML) pages, JavaScript object notation (JSON) files and messages, etc.). The server application (115) includes the interface controller (122), which processes the request (118) using the result graphs A (120).

The request (118) is a request from one of the user devices A (102) and B (107) through N (109). In one embodiment, the request (118) is a request for information about one or more entities defined in the ontology library (152), described in the file data (155), and graphed in the graph data (158). In one embodiment, the request (118) may specify additional filters for the list of entities. The structured text below (formatted in accordance with JSON) provides an example of entities that may be specified in the request (118) using key value pairs.

```
{
  “entity type”: “protein”,
  “entity”: “BRD9”,
}
{
  “entity type”: “disease”,
  “entity”: “breast cancer”,
}
```

The result graphs A (120) are generated with the modeling application (128), described further below. The result graphs A (120) includes nodes and edges in which the nodes correspond to text from the file data (155) and the edges correspond to semantic relationships between the nodes. The result graphs A (120) are directed graphs in which the edges identify a direction from one node to a subsequent node in the result graphs A (120). In one embodiment, the result graphs A (120) are acyclic graphs.

The interface controller (122) is a collection of programs that may operate on the server (112). The interface controller (122) processes the request (118) using the result graphs A (120) to generate the response (125). In one embodiment, the interface controller (122) searches the graph data (158) to identify the result graphs A (120) (which may include some of the graphs from the result graphs B (135)) that include information about the entities identified in the request (118).

The response (125) is generated by the interface controller (122) in response to the request (118) using the result graphs A (120). In one embodiment, the response (125) includes the result graphs A (120) and images from the file data (155) that correspond to the result graphs A (120). The response (125) may be a string of structured text (e.g., JSON (JavaScript object notation) text) that uses keys and values to store data. The contents of the response (125) may include the result graphs A (120), images, links to images, sentences from the file data (155), etc. The links to images may be in the form of uniform resource locators (URLs). The images may correspond to the result graphs A (120). The sentences may be the sentences used to generate the result graphs A (120). Portions of the response (125) may be displayed by the user devices A (102) and B (107) through N (109) that receive the response (125).

The modeling application (128) is a collection of programs that may operate on the server (112). The modeling application (128) generates the result graphs B (135) from the files (130) using a graph controller (132).

The files (130) include biomedical information and form the basis for the result graphs B (135). The files, which (130) include but are not limited to scientific publications, patents, internal evidence from experiments and summary documents (131), are the basis for the result graph (137). For example, each file includes multiple sentences and may include multiple images describing evidence of experiments. The evidence may identify how different entities, defined in the ontology library (152), affect each other. Entities that are proteins may suppress or enhance the expression of other entities and affect the prevalence of certain diseases. Types of entities include proteins, genes, diseases, experiment techniques, chemicals, cell lines, pathways, tissues, cell types, organisms, etc. In one embodiment, nouns and verbs from the sentences of the files (131) are mapped to the nodes (138) of the result graph (137). In one embodiment, the semantic relationships between the words in the sentences corresponding to the nodes (138) are mapped to the edges (140). In one embodiment, one file serves as the basis for multiple graphs. In one embodiment, one sentence from a file may serve as the basis for one graph.

The graph controller (132) generates the result graphs B (135) from the files (130) containing biomedical information. The graph controller (132) is a collection of programs that may operate on the server (112). For a sentence of the file (131), the graph controller (132) identifies the nodes (138) and the edges (140) for the result graph (137).

The result graphs B (135) are generated from the files (130) and include the result graph (137), which corresponds to the file (131), which includes biomedical information from an originating scientific publication. The nodes (138) represent nouns and verbs from a sentence of the file (131). The edges (140) identify semantic relationships between the words represented by the nodes (138).

The user devices A (102) and B (107) through N (109) are computing systems (further described in FIG. 7A). For example, the user devices A (102) and B (107) through N (109) may be desktop computers, mobile devices, laptop computers, tablet computers, server computers, etc. The user devices A (102) and B (107) through N (109) include hardware components and software components that operate as part of the system (100). The user devices A (102) and B (107) through N (109) communicate with the server (112) to access, manipulate, and view information including information (e.g., source ranks) from the graph data (158) and the file data (155). The user devices A (102) and B (107) through N (109) may communicate with the server (112) using standard protocols and file types, which may include hypertext transfer protocol (HTTP), HTTP secure (HTTPS), transmission control protocol (TCP), internet protocol (IP), hypertext markup language (HTML), extensible markup language (XML), etc. The user devices A (102) and B (107) through N (109) respectively include the user applications A (105) and B (108) through N (110).

The user applications A (105) and B (108) through N (110) may each include multiple programs respectively running on the user devices A (102) and B (107) through N (109). The user applications A (105) and B (108) through N (110) may be native applications, web applications, embedded applications, etc. In one embodiment, the user applications A (105) and B (108) through N (110) include web browser programs that display web pages from the server (112). In one embodiment, the user applications A (105) and B (108) through N (110) provide graphical user interfaces that display information stored in the repository (150).

As an example, the user application A (105) may be operated by a user and generate the request (118) to view information related to entities defined in the ontology library (152), described in the file data (155), and graphed in the graph data (158). Corresponding sentences and images from the file data (155) and graphs from the graph data (158) may be received in the response (125) and displayed in a user interface of the user application A (105).

As another example, the user device N (109) may be used by a developer to maintain the software applications hosted by the server (112) and train the machine learning models used by the system (100). Developers may view the data in the repository (150) to correct errors or modify the application served to the users of the system (100).

The repository (150) is a computing system that may include multiple computing devices in accordance with the computing system (700) and the nodes (722) and (724) described below in FIGS. 7A and 7B. The repository (150) may be hosted by a cloud services provider that also hosts the server (112). The cloud services provider may provide hosting, virtualization, and data storage services as well as other cloud services and to operate and control the data, programs, and applications that store and retrieve data from the repository (150). The data in the repository (150) includes the ontology library (152), the file data (155), the model data (157), and the graph data (158).

The ontology library (152) includes information that of the system (100). The entities and biomedical terms and phrases. Multiple terms and phrases may be used for the same entity. The ontology library (152) defines types of entities. In one embodiment, the types include the types of protein/gene, chemical, cell line, pathway, tissue, cell type, disease, organism, etc. The ontology library (152) may store the information about the entities in a database, structured text files, combinations thereof, etc.

The file data (155) includes biomedical information stored in text, images, data, etc. The biomedical information may include biomedical literature, patents, electric lab notebook (ELN) data, summary documents, etc., stored as electronic records. The biomedical information may be extracted from non-standard data sources, including tables, spreadsheets with data, genomic screening data, etc. The biomedical information describes the entities and corresponding relationships that are defined and stored in the ontology library (152). The file data (155) includes the files (130). Each file in the file data (155) may include image data and text data. The image data includes images that represent the graphical figures from the files. The text data represents the writings in the file data (155). The text data for a file includes multiple sentences that each may include multiple words that each may include multiple characters stored as strings in the repository (150). In one embodiment, the file data (155) includes biomedical information stored as extensible markup language (XML) files, portable document files (PDFs). The file formats define containers for the text and images of the biomedical information describing evidence of experiments.

The model data (157) includes the data for the models used by the system (100). The models may include rules-based models and machine learning models. The machine learning models may be updated by training, which may be supervised training. The modeling application (128) may load the models from the model data (157) to generate the result graphs B (135) from the files (130).

The model data (157) may also include intermediate data. The intermediate data is data generated by the models during the process of generating the result graphs B (135) from the files (130).

The graph data (158) is the data of the graphs (including the result graphs A (120) and B (135)) generated by the system. The graph data (158) includes the nodes and edges for the graphs. The graph data (158) may be stored in a database, structured text files, combinations thereof, etc.

Although shown using distributed computing architectures and systems, other architectures and systems may be used. In one embodiment, the server application (115) may be part of a monolithic application that implements evidence result networks. In one embodiment, the user applications A (105) and B (108) through N (110) may be part of monolithic applications that implement evidence result networks without the server application (115).

Turning to FIG. 1B, the graph controller (132) processes the file (131) to generate the result graphs B (135). In one embodiment, the graph controller includes the sentence controller (160), the token controller (162), the tree controller (164), and the text graph controller (167) to process the text from the file (131) describing evidence of biomedical experiments. In one embodiment, the graph controller includes the image controller (170), the text controller (172), and the image graph controller (177) to process the figures from the file (131) that provide evidence for the conclusions of the experiments.

The sentence controller (160) is a set of programs that operate to extract the sentences (161) from the file (131). In one embodiment, the sentence controller (160) cleans the text of the file (131) by removing markup language tags, adjusting capitalization, etc. The sentence controller (160) may split a string of text into substrings with each substring being a string that includes a sentence from the original text of the file (131). In one embodiment, the sentence controller (160) may filter the sentences and keep sentences with references to the figures of the file (131).

The sentences (161) are text strings extracted from the file (131). A sentence of the sentences (161) may be stored as a string of text characters. In one embodiment, the sentences (161) are stored in a list that maintains the order of the sentences (161) from the file (131). In one embodiment, the list may be filtered to remove sentences that do not contain a reference to a figure.

The token controller (162) is a set of programs that operate to locate the tokens (163) in the sentences (161). The token controller (162) may identify the start and stop of each token in a sentence

The tokens (163) identify the boundaries of words in the sentences (161). In one embodiment, a token (of the tokens (163)) may be a substring of a sentence (of the sentences (161)). In one embodiment, a token (of the tokens (163)) may be a set of identifiers that identify the locations of a start character and a stop character in a sentence. Each sentence may include multiple tokens.

The tree controller (164) is a set of programs that operate to generate the trees (165) from the tokens (163) of the sentences (161) of the file (131). In one embodiment, the tree controller (164) uses a neural network (e.g., the Berkeley Neural Parser)

The trees (165) are syntax trees of the sentences (161) to identify the parts of speech of the tokens (163) within the sentences (161). In one embodiment, the trees (165) are graphs with edges identifying parent child relationships between the nodes of a graph. In one embodiment, the nodes of a graph of a tree include a root node, intermediate nodes, and leaf nodes. The leaf nodes correspond to tokens (words, terms, multiword terms, etc.) from a sentence and the intermediate nodes identify parts of speech of the leaf nodes.

The text graph controller (167) is a set of programs that operate to generate the result graphs B (135) from the trees (165). In one embodiment, the text graph controller (167) maps the tokens (163) from the sentences (161) that represent nouns and verbs to nodes of the result graphs B (135). In one embodiment, the text graph controller (167) maps parts of speech identified by the trees (165) to the edges of the result graphs B (135).

In one embodiment, after generating an initial graph (of the result graphs B (135)) for a sentence (of the sentences (161)), the text graph controller (167) processes the graph using the ontology library (152) to identify the entities and corresponding entity types represented by the nodes of the graph. For example, a node of the graph may correspond to the token “BRD9”. The graph controller (167) identifies the token as an entity defined in the ontology library (152) and identifies the entity type as a protein.

The image controller (170) is a set of programs that operate to extract figures from the file (131) to generate the images (171). The image controller also extracts the figure text (169) that corresponds to the images (171). In one embodiment, the image controller (170) may use rules and logic to identify the images and corresponding image text from the file (131). In one embodiment, the image controller (170) may use machine learning models to identify the images (171) and the figure text (169). For example, the file (131) may be stored in a page friendly format (e.g., a portable document file (PDF)) in which each page of the publication is stored as an image in the file (131). A machine learning model may identify pages that include figures and the locations of the figures on those pages. The located figures may be extracted as the images (171). Another machine learning model may identify the legend text that corresponds to and describes the figures, which is extracted as the figure text (169).

The images (171) are image files extracted from the file (131). In one embodiment, the file (131) includes the figures as individual image files that the image controller (170) converts to the images (171). In one embodiment, the figures of the file (131) may be contained within larger images, e.g., the image of a page of the file (131). The image controller (170) processes the larger images to extract the figures as the images (171).

The figure text (169) is the text from the file (131) that describes the images (171). Each figure of the file (131) may include legend text that describes the figure. The legend text for one or more figures of the file (131) is extracted as the figure text (169), which corresponds to the images (171).

The text controller (172) is a set of programs that operate to process the images (171) and the figure text (169) to generate the structured text (173). The text controller (172) is further described with FIG. 1C below.

The structured text (173) is strings of nested text with information extracted from the images (171) using the figure text (169). In one embodiment, the structured text (173) includes a JSON formatted string for each image of the images (171). In one embodiment, the structured text (173) identifies the locations of text, panels, and experiment metadata within the images (171). In one embodiment, the structured text (173) includes text that is recognized from the images (171). The structured text (173) may include additional metadata about the images (171). For example, the structured text may identify the types of experiments and the types of techniques used in the experiments that are depicted in the images (171).

The image graph controller (177) is a set of programs that operate to process the structured text (173) to generate one or more of the result graphs B (135). In one embodiment, the graph controller (177) identifies text that corresponds to entities defined in the ontology library (152) from the structured text (173) and maps the identified text to nodes of the result graphs B (135). In one embodiment, the graph controller (177) uses the nested structure of the structure document (173) to identify the relationships between the nodes of one or more of the result graphs (135) and maps the relationships to edges of one or more of the result graphs B (135).

The result graphs B (135) are the graphs generated from the file (131) by the graph controller (132). The result graphs B (135) include nodes that represent entities defined in the ontology library (152) and include edges that represent relationships between the nodes.

The ontology library (152) defines the entities that may be recognized by the graph controller (132) from the file (131). The entities defined by the ontology library (152) are input to the token controller (162), the text graph controller (167), and the image graph controller (177), which identify the entities within the text and image extracted from the file (131).

Turning to FIG. 1C, the text controller (172) processes the image (180) and the corresponding legend text (179) to generate the image text (188). The text controller (172) may operate as part of the graph controller (132) of FIG. 1B.

The image (180) is one of the images (171) from FIG. 1B. The image (180) includes a figure from the file (131) of FIG. 1B.

The legend text (179) is a string from the figure text (169) of FIG. 1B. The legend text (179) is the text from the legend of the figure that corresponds to the image (180).

The text detector (181) is a set of programs that operate to process the image (180) to identify the presence and location of text within the image (180). In one embodiment, the text detector (181) uses machine learning models to identify the presence and location of text. The location may be identified with a bounding box that specifies four points of a rectangle that surrounds text that has been identified in the image (180). The location of the text from the text detector (181) may be input to the text recognizer (182).

The text recognizer (182) is a set of programs that operates to process the image (180) to recognize text within the image (180) and output the text as a string. The text recognizer (182) may process a sub image from the image (180) that corresponds to a bounding box identified by the text detector (181). A machine learning model may then be used to recognize the text from the sub image and output a string of characters that correspond to the text within the sub image.

The panel locator (183) is a set of programs that operates to process the image (180) to identify the location of panels and subpanels within the image (180) or a portion of the image (180). A panel of the image (180) is a portion of the image, which may depict evidence of an experiment. The panels of the image (180) may contain subpanels to further subdivide information contained within the image (180). The image (180) may include multiple panels and subpanels that may identified within the legend text (179). The panel locator (183) may be invoked to identify the location for each panel (or subpanel) identified in the legend text (179). In one embodiment, the panel locator (183) outputs a bit array with each bit corresponding to a pixel from the image (180) and identifying whether the pixel corresponds to a panel.

The experiment detector (184); is a set of programs that operates to process the image (180) to identify metadata about experiments depicted in the image (180). In one embodiment, the experiment detector (184) processes the image (180) with a machine learning model (e.g., a convolutional neural network) that outputs a bounding box and a classification. In one embodiment, the bounding box may be an array of coordinates (e.g., top, left, bottom, right) in the image that identify the location of evidence of an experiment within the image. In one embodiment, the classification may be a categorical value that identifies experiment metadata, which may include the type of evidence, the type of experiment, or technique used in the experiment (e.g., graph, western blot, etc.).

The text generator (185) is a set of programs that operate to process the outputs from the text detector (181), the text recognizer (182), the panel locator (183), and the experiment detector (184) to generate the image text (188). In one embodiment, the text generator (185) creates a nested structure for the image text (188) based on the outputs from the panel locator (183), the experiment detector (184), and the text detector (181). For example, the text generator (185) may include descriptions for the panels, experiment metadata, and text from the image (180) in which the text and description of the experiment metadata may be nested within the description of the panels. Elements for subpanels may be nested within the elements for the panels.

The image text (188) is a portion of the structured text (173) of FIG. 1B that corresponds to the image (180). In one embodiment, the image text (188) uses a nested structure to describe the panels, experiment metadata, and text that are identified and located within the image (180).

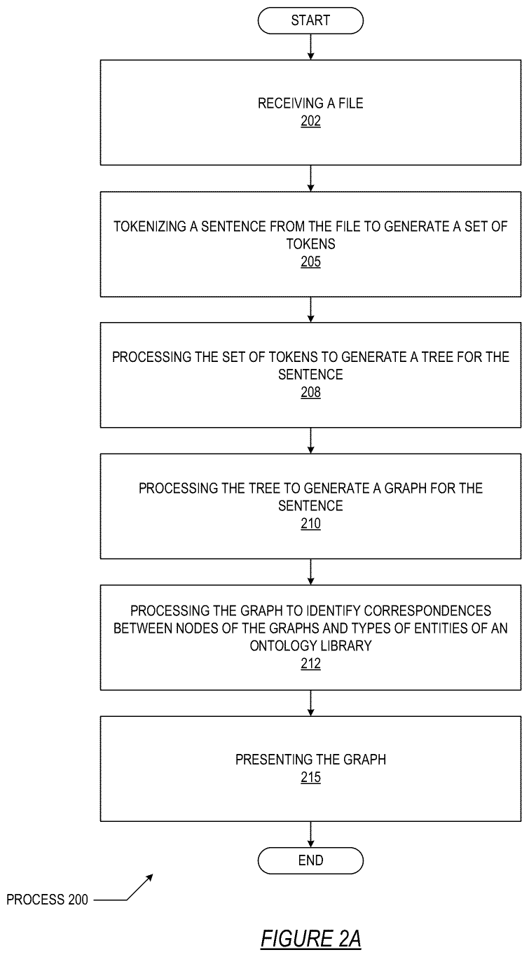

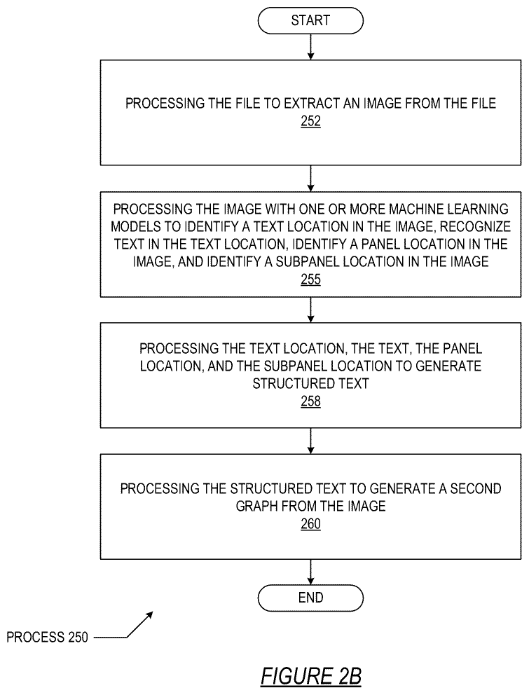

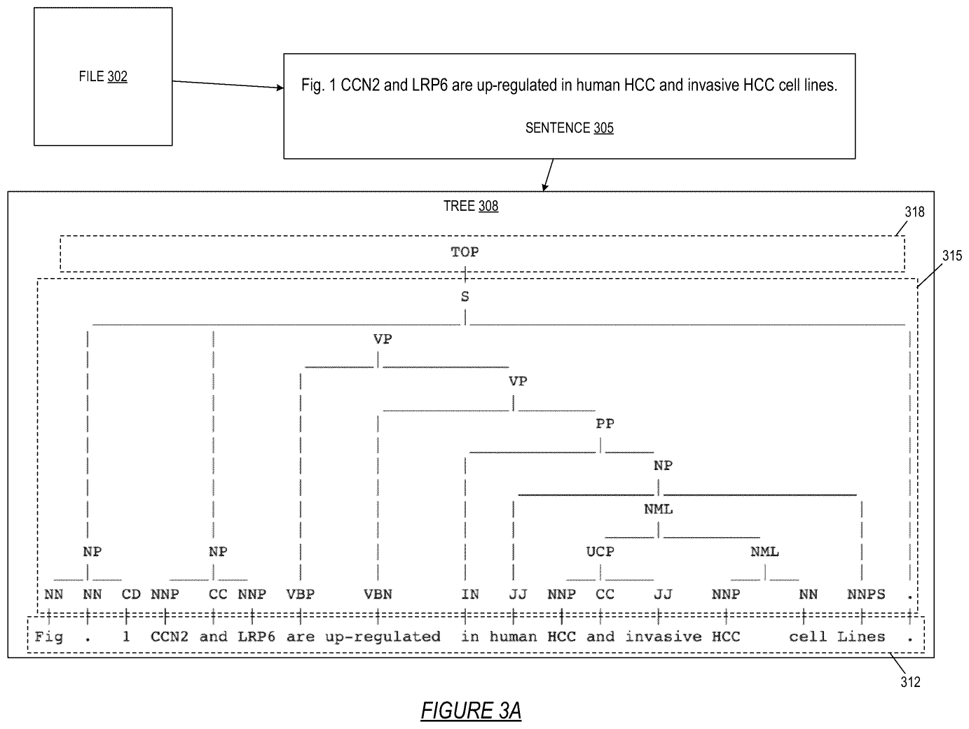

Turning to FIG. 2A, the process (200) implements evidence result networks. The process (200) may be performed by a computing system, such as the computing system (700) of FIG. 7A.

At Step 202, a file with biomedical information is received. The file may be one of multiple files stored in a repository. The files in the repository may be periodically updated to include new files. In one embodiment, the new files are available through third party websites.

In one embodiment, the file is filtered to retain the sentence when the sentence corresponds to a figure of the file. In one embodiment, sentences that do not correspond to a figure may be removed from a list of sentences being processed by the system. A computing system may identify a sentence as corresponding to a figure when the sentence includes an identification of the figure. For example, the sentence:

“FIG. 1 shows the protein BRD9.”

corresponds to “FIG. 1” of a publication. Using the regular expression:

“/Figure \d+/”

a computing system may identify that the sentence corresponds to a figure of the file.

In one embodiment, a machine learning model may be used to identify the correspondence. The machine learning model may receive the sentence as an input and outputs a classification of whether a sentence corresponds to a figure and may identify the figure to which this sentence corresponds.

In one embodiment, the file is processed to extract the sentence from the file. The sentence may be extracted by opening a file containing the text of the sentences of the file, and splitting the text based upon sentence boundaries (e.g., the character “.”) into a list that includes elements for the sentences from the file. A sentence may be stored as a string of characters to a repository.

At Step 205, a sentence from the file is tokenized to generate a set of tokens. In one embodiment, a token is stored as a substring of the string. The token may be stored by saving the characters from the sentence to a string that forms the token. In one embodiment, the token may identify the start character and stop character in the sentence for the location of the token within the sentence from which the substring may be extracted. In one embodiment, a token is a multiword entity defined in an ontology library corresponding to a biomedical meaning A multiword entity is a set of one or more words that refers to a single entity. For example, “breast cancer” is a multiword entity that refers to the disease of cancer in the breast.

At Step 208, the set of tokens is processed to generate a tree for the sentence. The set of tokens for a sentence may be input to a semantic parser that generates the tree. In one embodiment, the tree generated from a sentence includes a root node, intermediate nodes, and leaf nodes. The leaf nodes correspond to tokens from the sentence and the intermediate nodes identify parts of speech of the leaf nodes.

At Step 210, the tree is processed to generate a graph for the sentence. In one embodiment, the nouns and verbs of the sentence that are identified by the tree are used to form the nodes of the graph. In one embodiment, the semantic relationships between the nouns and verbs that are identified by the tree are used to form the edges of the graph.

At Step 212, the graph is processed to identify correspondences between nodes of the graphs and types of entities of an ontology library. In one embodiment, the correspondence is identified by matching a token (i.e., a substring of a sentence) to corresponding to a node to the words defined as representing an entity in the ontology library. The entity may be represented by a string stored in the ontology library. In one embodiment, the tokens of the sentence represented by the graph are input to a machine learning model. The machine learning model may be a neural network, which may include a transformer network, a recurrent neural network, a fully connected neural network, an autoencoder, etc. The output of the machine learning model may be a classification of the tokens of the sentence to the types of entities recognized by the system.

Multiple machine learning models may be used to generate a graph from a file. The file may be from ELNs, portfolio documents, scientific data in raw/table form, presentations, etc., and may be from private internal data. The system may federate public data with private internal data to generate graphs from the public and private data. The different machine learning models may be trained with supervised learning with labels for inputs that are compared to the outputs during training. The different between the outputs and the labels are fed back to the machine learning model to update the parameters and weights of the machine learning models.

At Step 215, the graph is presented. The graph may be presented by transmitting the graph to a user device as a response to a request from the user device. The user device may display the graph in a user interface that shows the nodes and edges of the graph. The nodes of the graph may have different colors or shapes that identify the entity types of the nodes. For example, nodes that represent proteins may be displayed as a circle, nodes that represent diseases may be shaped with a square, etc.

In one embodiment, the graph is presented with an image from the file. The image corresponds to a figure identified by the sentence represented by the graph.

Turning to FIG. 2B, the process (250) implements evidence result networks by generating graphs from images of a file. The process (250) may be performed by a computing system, such as the computing system (700) of FIG. 7A.

At Step 252, the file is processed to extract an image from the file. In one embodiment, the images may be extracted by copying image files that correspond to the images of a publication.

In one embodiment, the images may be extracted from a file that includes pages of a publication. For example, a machine learning model may receive an image of a page of the publication and output the location of a figure within the image of the page from the file. The image of the page from the file may be cropped using the location of the figure to generate an image of the figure.

At Step 255, the image is processed with one or more machine learning models to identify a text location in the image, recognize text in the text location, identify a panel location in the image, and identify experiment metadata in the image. In one embodiment, different machine learning models are used to generate the text locations, recognized text, panel locations, and experiment metadata.

At Step 258, the text location, the text, the panel location, and the experiment metadata are processed to generate structured text. In one embodiment, the text location, experiment metadata, and panel locations are used to create nested structures in the text. For example, the structured text may include a list with elements for the panels. An element for a panel may include a list with elements for experiment metadata and a list with elements for text. The text elements may include the text recognized from the image. The image panels may include subpanels and the image panels and subpanels may be processed with machine learning models that recognize and classify the subpanels, panels, image, text, experiment metadata, etc. as entities or entity types defined in the ontology library. For example, a panel may show evidence of an experiment using a western blot. A machine learning model may classify the panel as having an entity of “western blot” and an entity type of “technique”. The entity and entity type for the panels and subpanels may be recorded in the structured text for the image.

At Step 260, the structured text is processed to generate a second graph from the image. In one embodiment, the structured text may be converted to a tree. For example, the nesting of lists in the structured text may identify parent child relationships between nodes for the panels, subpanels, text, etc. The tree from the structured text may then be processed to generate a graph similar to the graphs generated from the sentences of the file. The nodes of the graph generated from the tree generated from the image may represent the nouns of verbs recognized from the text of the image. The edges of the graph may identify relationships between the leaf nodes based on the entity types and based on the entities represented by the nodes of the tree.

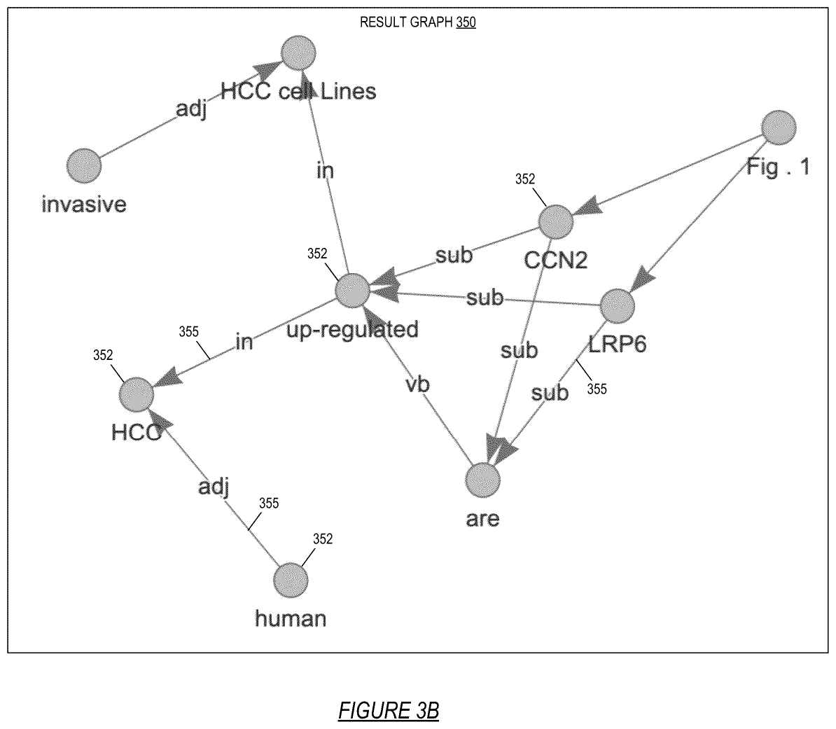

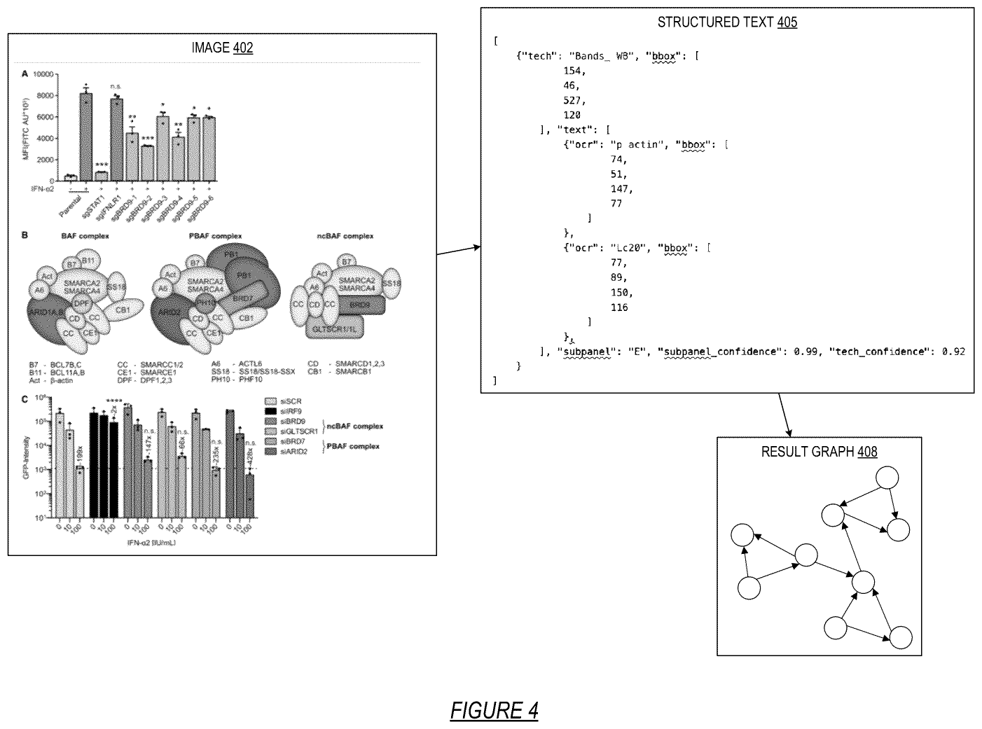

Turning to FIG. 3A, the file (302) is shown from which the sentence (305) is extracted, which is used to generate the tree (308), which is used to generate the graph (350) (of FIG. 3B). The file (302), the sentence (305), the tree (308), and the result graph (350) (of FIG. 3B) may be stored as electronic files, transmitted in messages, and displayed on a user interface.

The file (302) is a collection of biomedical information, which may include, but is not limited to, a writing of biomedical literature with sentences and figures stored as text and images. Different sources of biomedical information may be used. The file (302) is processed to extract the sentence (305).

The sentence (305) is a sentence from the file (302). The sentence (305) is stored as a string of characters. In one embodiment, the sentence (305) is tokenized to identify the locations of entities within the sentence (305). For example, the entities recognized from the sentence (305) may include “CCN2”, “LRP6”, “HCC”, and “HCC cell lines”. The sentence (305) is processed to generate the tree (308).

The tree (308) is a data structure that identifies semantic relationships of the words of the sentence (305). The tree (308) includes the leaf nodes (312), the intermediate nodes (315), and the root node (318).

The leaf nodes (312) correspond to the words from the sentence (305). The leaf nodes have no child nodes. The leaf nodes have parent nodes in the intermediate nodes (315).

The intermediate nodes (315) include values that identify the parts of speech of the leaf nodes (312). The intermediate nodes (315) having leaf nodes as direct children nodes identify the parts of speech of the words represented by the leaf nodes. The intermediate nodes (315) that do not have leaf nodes as direct children nodes identify the parts of speech of groups of one or more words, i.e., phrases, of the sentence (305).

The root node (318) is the top of the tree (308). The root node (318) has no parent node.

Turning to FIG. 3B, the result graph (350) is a data structure that represents the sentence (305) (of FIG. 3A). The result graph (350) may be generated from the sentence (305) and the tree (308). The nodes of the result graph (350) represent nouns (e.g., “CCN2”, “HCC”, etc.) and verbs (e.g., “up-regulated”, “are”, etc.) from the sentence (305). The edges (355) identify semantic relationships (e.g., subject “sub”, verb “vb”, adjective “adj”) between the words of the nodes (352) of the sentence (305) (of FIG. 3A). The result graph (350) is a directed acyclic graph.

Turning to FIG. 4, the image (402) is shown from which the structured text (405) is generated, which is used to generate the result graph (408). The image (402), the structured text (405), and the result graph (408) may be stored as electronic files, transmitted in messages, and displayed on a user interface.

The image (402) is a figure from a file (e.g., the file (302) of FIG. 3A, which may be from a biomedical publication). In one embodiment, the image (402) is an image file that is included with or as part of the file (302) of FIG. 3A. In one embodiment, the image (402) is extracted from an image of a page of a publication stored as the file (302) of FIG. 3A. The image (402) includes three panels labeled “A”, “B”, and “C”. The “B” panel includes three subpanels labeled “BAF complex”, “PBAF complex”, and “ncBAF complex”. The image (402) is processed to recognize the locations of the panels, subpanels, and text using machine learning models. After being located, the text from the image is recognized and stored as text (i.e., strings of characters). The panel, subpanel, and text locations along with the recognized text are processed to generate the structured text (405).

The structured text (405) is a string of text characters that represents the image (402). In one embodiment, the structured text (405) includes nested lists that form a hierarchical structure patterned after the hierarchical structure of the panels, subpanels, and text from the image (402). The structured text (405) is processed to generate the result graph (408).

The result graph (408) is a data structure that represents the figure, corresponding to the image (402), from a file (e.g., the file (302) of FIG. 3A). The result graph (408) includes nodes and edges. The nodes represent nouns and verbs identified in the structured text (405). The edges may represent the nested relationships between the panels, subpanels, and text of the image (402) described in the structured text (405).

Turning to FIG. 5, the tagged sentence (502) is generated from a sentence and used to generate the updated result graph (505). The tagged sentence (502) and the updated result graph (505) may be stored as electronic files, transmitted in messages, and displayed on a user interface.

The tagged sentence (502) is a sentence from a file that has been processed to generate the updated result graph (505). The sentence from which the tagged sentence is derived is input to a model to tag the entities in the sentence to generate the tagged sentence (502). The model may be a rules-based model, an artificial intelligence model, combinations thereof, etc.

As an example, the underlined portion (“INSR and PIK3R1 levels were not altered in TNF-alpha treated myotubes”) is tagged by the model. The terms “INSR”, “PIK3R1”, and “TNF-alpha” may be tagged as one type of entity that is presented as green when displayed on a user interface. The term “not” is tagged and may be displayed as orange. The terms “altered” and “treated” are tagged and may be displayed as pink. The term “myotubes” is tagged and may be displayed as red. After being identified in the sentence, the tags may be applied to the graph to generate the updated result graph (505).

The updated result graph (505) is an updated version of a graph of the sentence used to generate the tagged sentence (502). The graph is updated to label the nodes of the graph with the tags from the tagged sentence. For example, the nodes corresponding to “INSR” and “PIK3R1” are labeled with tags identified in the tagged sentence and may be displayed as green. The node corresponding to “altered” is tagged and displayed as pink. The node corresponding to “myotubes” is tagged and displayed as red.

Turning to FIG. 6, the user interface (600) displays information from a file, which may be a publication of biomedical literature. Different sources of files may be used. The user interface (600) may display the information on a user device after receiving a response to a request for the information transmitted to a server application. For example, the request may be for a publication that includes evidence linking the proteins “BRD9” and “A549”. The user interface displays the header section (602), the summary section (605), and the figure section (650).

The header section (602) includes text identifying the file being displayed. In one embodiment, the text in the header section (602) includes the name of the publication, the name of the author, the title of the publication, etc., which may be extracted from the file. Additional sources of information may be used, including patents, ELN data, summary documents, portfolio documents, scientific data in raw/table form, presentations, etc., and similar information may be extracted.

The summary section (605) displays information from the text of the file identified in the header section (602). The summary section (605) includes the graph section (608) and the excerpt section (615).

The graph section (608) includes the result graphs (610) and (612). the result graphs (610) and (612) were generated from the sentence displayed in the excerpt section (615). The result graph (612) shows the link between the proteins “BRD9” and “A549”, which conforms to the request that prompted the response with the information displayed in the user interface (600).

The excerpt section (615) displays a sentence from the file identified in the header section (602). The sentence in the excerpt section (615) is the basis from which the result graphs (610) and (612) were generated by tokenizing the sentence, generating a tree from the tokens, and generating the result graphs (610) and (612) from the tokens and tree.

The figure section (650) displays information from the figures of the file identified in the header section (602). The figure section (650) includes the image section (652) and the legend section (658).

The image section (652) displays the image (655). The image (655) was extracted from the file identified in the header section (602). The image (655) corresponds to the text from the legend section (658). The image (655) corresponds to the result graph (612) because the sentence shown in the excerpt section (615) identifies the figure (“Fig EV1A”) that corresponds to the image (655).

The legend section (658) displays the text of the legend that corresponds to the figure of the image (655). In one embodiment, the text of the legend section (658) may be processed to generate one or more graphs from the sentence in the legend section (658).

Embodiments of the invention may be implemented on a computing system. Any combination of a mobile, a desktop, a server, a router, a switch, an embedded device, or other types of hardware may be used. For example, as shown in FIG. 7A, the computing system (700) may include one or more computer processor(s) (702), non-persistent storage (704) (e.g., volatile memory, such as a random access memory (RAM), cache memory), persistent storage (706) (e.g., a hard disk, an optical drive such as a compact disk (CD) drive or a digital versatile disk (DVD) drive, a flash memory, etc.), a communication interface (712) (e.g., Bluetooth interface, infrared interface, network interface, optical interface, etc.), and numerous other elements and functionalities.

The computer processor(s) (702) may be an integrated circuit for processing instructions. For example, the computer processor(s) (702) may be one or more cores or micro-cores of a processor. The computing system (700) may also include one or more input device(s) (710), such as a touchscreen, a keyboard, a mouse, a microphone, a touchpad, an electronic pen, or any other type of input device.

The communication interface (712) may include an integrated circuit for connecting the computing system (700) to a network (not shown) (e.g., a local area network (LAN), a wide area network (WAN) such as the Internet, a mobile network, or any other type of network) and/or to another device, such as another computing device.

Further, the computing system (700) may include one or more output device(s) (708), such as a screen (e.g., a liquid crystal display (LCD), a plasma display, a touchscreen, a cathode ray tube (CRT) monitor, a projector, or other display device), a printer, an external storage, or any other output device. One or more of the output device(s) (708) may be the same or different from the input device(s) (710). The input and output device(s) (710 and (708)) may be locally or remotely connected to the computer processor(s) (702), non-persistent storage (704), and persistent storage (706). Many different types of computing systems exist, and the aforementioned input and output device(s) (710 and (708)) may take other forms.

Software instructions in the form of computer readable program code to perform embodiments of the invention may be stored, in whole or in part, temporarily or permanently, on a non-transitory computer readable medium such as a CD, a DVD, a storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (700) in FIG. 7A may be connected to or be a part of a network. For example, as shown in FIG. 7B, the network (720) may include multiple nodes (e.g., node X (722), node Y (724)). Each node may correspond to a computing system, such as the computing system (700) shown in FIG. 7A, or a group of nodes combined may correspond to the computing system (700) shown in FIG. 7A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (700) may be located at a remote location and connected to the other elements over a network.

Although not shown in FIG. 7B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (722), node Y (724)) in the network (720) may be configured to provide services for a client device (726). For example, the nodes may be part of a cloud computing system. The nodes may include functionality to receive requests from the client device (726) and transmit responses to the client device (726). The client device (726) may be a computing system, such as the computing system (700) shown in FIG. 7A. Further, the client device (726) may include and/or perform all or a portion of one or more embodiments of the invention.

The computing system (700) or group of computing systems described in FIGS. 7A and 7B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data sharing techniques described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a graphical user interface (GUI) on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system (700) in FIG. 7A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail-such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system (700) of FIG. 7A, while performing one or more embodiments of the invention, may perform data comparison. Data comparison may be used to compare two or more data values (e.g., A, B). For example, one or more embodiments may determine whether A>B, A=B, A!=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system (700) in FIG. 7A may implement and/or be connected to a data repository. For example, one type of data repository is a database. A database is a collection of information configured for ease of data retrieval, modification, re-organization, and deletion. A Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g., join, full join, count, average, etc.), sort (e.g., ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system (700) of FIG. 7A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system (700) of FIG. 7A and the nodes (e.g., node X (722), node Y (724)) and/or client device (726) in FIG. 7B. Other functions may be performed using one or more embodiments of the invention.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method comprising:
receiving a file;
tokenizing a sentence from the file to generate a set of tokens;
processing the set of tokens to generate a tree for the sentence;
processing the tree to generate a graph for the sentence;
processing the graph to identify correspondences between nodes of the graphs and types of entities of an ontology library; and
presenting the graph.

2. The method of claim 1, further comprising:
presenting the graph with an image from the file.

3. The method of claim 1, further comprising:
processing the file to extract an image from the file;
processing the image with one or more machine learning models to identify a text location in the image, recognize text in the text location, identify a panel location in the image, and identify experiment metadata in the image;
processing the text location, the text, the panel location, and the experiment metadata to generate structured text; and
processing the structured text to generate a second graph from the image.

4. The method of claim 1, further comprising:
processing the graph by tagging the nodes to identify entity types of the nodes, wherein the entity types are defined by the ontology library.

5. The method of claim 1, further comprising:
filtering the file to retain the sentence when the sentence corresponds to a figure of the file.

6. The method of claim 1, further comprising:
processing the file to extract the sentence from the file;
storing the sentence as a string; and
storing a token as a substring of the string.

7. The method of claim 1, further comprising:
generating the set of tokens, wherein a token, of the set of tokens, is a multiword entity defined in the ontology library corresponding to a biomedical meaning.

8. The method of claim 1, further comprising:
generating the tree comprising a root node, intermediate nodes, and leaf nodes, wherein the leaf nodes correspond to tokens from the sentence and the intermediate nodes identify parts of speech of the leaf nodes.

9. The method of claim 1, further comprising:
generating the graph as a directed graph comprising one or more nodes corresponding to tokens of nouns and verbs identified from the tree and comprising one or more edges identifying semantic relationships between the nodes of the graphs.

10. The method of claim 1, further comprising:
training a machine learning model used by a modeling application to generate the graph from the file.

11. A system comprising:
a graph controller configured to generate a graph;
an application executing on one or more servers and configured for:
receiving a file,
tokenizing a sentence from the file to generate a set of tokens,
processing the set of tokens to generate a tree for the sentence,
processing the tree to generate the graph for the sentence,
processing the graph to identify correspondences between nodes of the graphs and types of entities of an ontology library, and
presenting the graph.

12. The system of claim 11, wherein the application is further configured for:
presenting the graph with an image from the file.

13. The system of claim 11, wherein the application is further configured for:
processing the file to extract an image from the file;
processing the image with one or more machine learning models to identify a text location in the image, recognize text in the text location, identify a panel location in the image, and identify experiment metadata in the image;
processing the text location, the text, the panel location, and the experiment metadata to generate structured text; and
processing the structured text to generate a second graph from the image.

14. The system of claim 11, wherein the application is further configured for:
processing the graph by tagging the nodes to identify entity types of the nodes, wherein the entity types are defined by the ontology library.

15. The system of claim 11, wherein the application is further configured for:
filtering the file to retain the sentence when the sentence corresponds to a figure of the file.

16. The system of claim 11, wherein the application is further configured for:
generating the set of tokens, wherein a token, of the set of tokens, is a multiword entity defined in the ontology library corresponding to a biomedical meaning.

17. The system of claim 11, wherein the application is further configured for:
generating the tree comprising a root node, intermediate nodes, and leaf nodes, wherein the leaf nodes correspond to tokens from the sentence and the intermediate nodes identify parts of speech of the leaf nodes.

18. The system of claim 11, wherein the application is further configured for:
generating the graph as a directed graph comprising one or more nodes corresponding to tokens of nouns and verbs identified from the tree and comprising one or more edges identifying semantic relationships between the nodes of the graphs.

19. The system of claim 11, wherein the application is further configured for:
training a machine learning model used by a modeling application to generate the graph from the file.

20. A method comprising:
transmitting a request;
displaying a graph received in a response to the request, wherein the graph is generated by:
tokenizing a sentence from a file to generate a set of tokens;
processing the set of tokens to generate a tree for the sentence;
processing the tree to generate a graph for the sentence; and
processing the graph to identify correspondences between nodes of the graphs and types of entities of an ontology library.
